SEQUENCE LISTING (2 sequences)

SEQ ID NO: 1 - DNA, 792 bp, Artificial sequence
  Synthetic oligonucleotide
gtctaagaaaccattattatcatgacattaacctataaaaataggcgtatcacgaggccctttcgtcttcactcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattcgagctcggtacccggggatcctctagtcagctgacgcgtgctagcgcggccgcatcgataagcttgtcgacgatatctctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcctcgagcttcctcgctcactgactcgctgcgctcggtcgttcggctgcggcgagcggtatcagctcactcaaaggcggtaatacggttatccacagaatcaggggataacgcaggaaagaa

SEQ ID NO: 2 - DNA, 657 bp, Sus scrofa
ccaccatggctgcaggccctcggacctccgtgctcctggctttcgccctgctctgcctgccctggactcaggaggtgggcgccttcccagccatgcccttgtccagcctatttgccaacgccgtgctccgggcccagcacctgcaccaactggctgccgacacctacaaggagtttgagcgcgcctacatcccggagggacagaggtactccatccagaacgcccaggctgccttctgcttctcggagaccatcccggcccccacgggcaaggacgaggcccagcagagatcggacgtggagctgctgcgcttctcgctgctgctcatccagtcgtggctcgggcccgtgcagttcctcagcagggtcttcaccaacagcctggtgtttggcacctcagaccgcgtctacgagaagctgaaggacctggaggagggcatccaggccctgatgcgggagctggaggatggcagcccccgggcaggacagatcctcaagcaaacctacgacaaatttgacacaaacttgcgcagtgatgacgcgctgcttaagaactacgggctgctctcctgcttcaagaaggacctgcacaaggctgagacatacctgcgggtcatgaagtgtcgccgcttcgtggagagcagctgtgccttctagt

(19) United States
(12) Patent Application Publication
(10) Pub. No.: US 2013/0053628 A1
(43) Pub. Date: Feb. 28, 2013

(54) METHOD FOR CULTIVATING A TRANSGENIC ANIMAL WITH INCREASED EXPRESSION AMOUNT OF PORCINE GROWTH HORMONE

(71) Applicants: Kui Li, Beijing (CN); Huiming Ju, Yangzhou City (CN); Xinmin Zheng, Wuhan City (CN); Lijing Bai, Beijing (CN); Wentao Cui, Beijing (CN); Zhonglin Tang, Beijing (CN); Yulian Mu, Beijing (CN); Shulin Yang, Beijing (CN)

(72) Inventors: Kui Li, Beijing (CN); Huiming Ju, Yangzhou City (CN); Xinmin Zheng, Wuhan City (CN); Lijing Bai, Beijing (CN); Wentao Cui, Beijing (CN); Zhonglin Tang, Beijing (CN); Yulian Mu, Beijing (CN); Shulin Yang, Beijing (CN)

(73) Assignee: Institute of Animal Science, Chinese Academy of Agricultural Sciences, Beijing (CN)

(21) Appl. No.: 13/122,400

(22) PCT Filed: Jun. 24, 2010
(86) PCT No.: PCT/CN10/00942

§ 371 Date: Sep. 27, 2012

(30) Foreign Application Priority Data
Jan. 27, 2010 (CN) 201010102427.4

(51) Int. Cl. C12N 15/85; A61B 17/435

(52) U.S. Cl. 600/34; 800/21

(57) ABSTRACT

A method for cultivating a transgenic animal with an increased expression amount of porcine growth hormone is provided. Additionally, a method for cultivating a transgenic embryo, which introduces a recombinant expression vector of 1) TRE-GH or 2) TET-ON into a target embryo to obtain a transgenic embryo with a higher expression amount of porcine growth hormone than a target embryo without introduction is provided. A transgenic animal can be obtained by transplanting the embryo into an animal. The experiments demonstrate that growth hormone (GH) is sharply increased in blood of transgenic pigs after adding DOX.

### TECHNICAL FIELD

The present invention relates to a method for cultivating a transgenic animal with increased expression amount of porcine growth hormone.

### BACKGROUND OF THE INVENTION

In 70s of the 20th century, Jaenich et al. introduced the DNA of SV40 into the blastula of mice, and detected SV40 DNA in the tissues of the progeny mice, demonstrating that it is possible to introduce an exogenous gene into an embryonic cell and achieve integration. In 80s of the 20th century, Gordon et al. developed the technique of microinjection for transgenic animal. From then on, this technique was developed rapidly with various methods for developing transgenic animals occurring in succession, and its use was infiltrated into numerous research fields, which became a powerful tool for people to understand genetic materials in organism deeply, learn gene functions, and establish animal models for human diseases to research diagnosis and treatment for the diseases. Although transgenic animals have an attractive prospect for use, in the transgenic positive animals obtained early, the transgene is expressed successively in the early stage of an embryo, which cannot ensure the tissue and/or developmental stage specificity of the transgene expression. When the gene researched has toxicity or lethal effect on embryonic development, the embryo will die at early stage of the development, so that the subsequent research cannot be performed. Thus, if the value for the use of a transgenic animal is to be achieved, the achievement of stage and tissue specificity of transgene expression will be a key issue. For this reason, on the basis of understanding the gene regulation mechanism for organism deeply, people constructed a series of systems that can regulate transgene expression, causing that the transferred gene can be expressed in a manner of time and space specificity according to the intention of the researcher. There are a number of regulation systems existed currently, wherein tetracycline regulation system (abbreviation: tet regulation system) is more researched and most broadly used. Using the system to prepare a transgenic domestic animal (pig, cattle and sheep) can achieve a timing expression and a timing integration of an exogenous gene in the body of a domestic animal, which is a new method for preparing a transgenic domestic animal capable of regulating the expression of an exogenous gene at any time.

### DISCLOSURE OF THE INVENTION

A purpose of the present invention is to provide a method for cultivating a transgenic embryo with increased expression amount of porcine growth hormone.

In the method for cultivating a transgenic embryo with increased expression amount of porcine growth hormone provided by the present invention, a recombinant expression vector as 1) or 2) below is introduced into a target embryo to obtain a transgenic embryo with higher expression amount of porcine growth hormone than said target embryo:

### 1) TRE-GH and TET-ON;

### 2) TRE-GH-TET-ON,

wherein, TRE-GH is a recombinant expression vector formed by inserting the DNA fragment presented as SEQ ID NO. 2 in the Sequence Listing into the polyclonal site of vector TRE which is a recombinant vector formed by inserting the DNA fragment presented as SEQ ID NO. 1 in the Sequence Listing into the polyclonal site of plasmid pUC19,
TRE-GH-TET-ON is a recombinant expression vector obtained by linking a fragment of 2.5 Kb in size obtained by the cleavage of TET-ON with two enzymes of NheI and HindIII to a fragment of 3.3 KB in size obtained by the cleavage of TRE-GH with two enzymes of NheI and HindIII.

Vector TET-ON is the abbreviation of pTet-On-Advanced, commercially available from Clontech Corporation, Catalog No. 630930. The aforementioned embryo is an embryo in pronuclear phase.

The aforementioned embryo may be an embryo of any one of animals except human. Specifically, it could be an embryo of a pig, cattle, sheep, cat, dog, rabbit or murine.

Another purpose of the present invention is to provide a method for cultivating a transgenic animal with increased expression amount of porcine growth hormone.

In the method for cultivating a transgenic animal provided in the present invention, the transgenic embryo prepared by the aforementioned method is transplanted into the body of a female target animal to obtain the transgenic animal with higher expression amount of porcine growth hormone than said targeted animal.

The target animal described above is any one of animals except human. Specifically, it could be a pig, cattle, sheep, cat, dog, rabbit or murine.

### DESCRIPTION OF THE DRAWINGS

FIG. 1: The profile of plasmid TRE-GH.

FIG. 2: The induced expression of GH at embryonic level.

FIG. 3: The PCR detection for genomic DNAs of TRE-GH and TET-ON transgenic pigs, wherein M is 100 bp ladder DNA mark; 1-23 are prepared porcine progenies to be detected; and + is a positive control (with plasmids TRE-GE and TET-ON as template).

FIG. 4: The semi-quantitative detection of the expressions of rtTA in TRE-GH and TET-ON transgenic pigs as well as in TRE-GH-TET-ON transgenic pigs. M is 100 bp ladder DNA mark; 1, 4, 7, 11, 13, 15, 18, 19 and 22 are TRE-GH and TET-ON transgenic pigs; 37, 38, 39, 43, 48, 49, 52, 55, 57 and 58 are TRE-GH-TET-ON transgenic pigs; Y1, Y2, Y3 and Y4 are non-transgenic pigs; and K is a blank control (amplified with water as template).

FIG. 5: The profile of TRE-GH-TET-ON.

FIG. 6: The PCR detection for genomic DNAs of TRE-GH-TET-ON transgenic pigs (M is 100 bp ladder DNA mark; 1-23 are prepared porcine progenies to be detected; and + is positive control (with TRE-GH-TET-ON as template)).

FIG. 7: The expression of GH in normal pigs and transgenic pigs induced by deoxytetracycline (DOX). 22, 37, 4 and 55 are indicative of GH expressions of different transgenic pigs not processed with deoxytetracycline; 22+DOX, 37+DOX et al. are indicative of GH expressions of different pigs processed with deoxytetracycline.

### PREFERRED EMBODIMENTS FOR CARRYING OUT THE INVENTION

Hereinafter, the present invention will be further described in combination with specific examples. However, the present invention is not limited to the following examples.

In the following examples, conventional methods are used, unless indicated otherwise.

### Example 1

### Obtainment of Transgenic Embryos

### I. Obtainment of Recombinant Expression Vectors

1. Obtainment of linearized TRE-GH and TET-ON

A DNA fragment containing a TRE promoter, a polyclonal site and a SV40 polyA (named as TRE-MSC-SV40 polyA gene, the nucleotide sequence of which is presented as SEQ ID NO. 1 in the Sequence Listing, synthesized by Beijing AuGCT Biotech Company) is synthesized. The synthesized DNA fragment described above is kept at 95° C. for 10 minutes, and then immediately placed into ice water. After the treatment, it is cleaved with two enzymes of Zral. (commercially available from NEB Corporation, Catalog No. R0659L) and Pcil (commercially available from NEB Corporation, Catalog No. R0655L) for use. Plasmid pUC19 (commercially available from Takara Biotechnology Co. Ltd., Catalog No. FD3219) is cleaved with two enzymes of Zral. and Pcil, and then is linked to aforementioned TRE-MSC-SV40 polyA fragment (the ligase is commercially available from promega corporation, Catalog No. M1804). The competent Escherichia coli DH5a (commercially available from Beijing TransGen Biotech Co., Ltd., Catalog No. CD201) is transformed according to the operating steps required by the manufacture. The DH5a liquid is coated onto an agarose gel plate, and cultivated at 37° C. in an incubator overnight. A single colony is picked and inoculated into a liquid LB medium for culture, and part of the DH5a liquid is sent to Invitrogen Beijing Office for sequencing. The vector with correct sequencing result (named as TRE vector) is selected for use.

The blood from auricular veins of pigs (large white pig strain, commercially available from Xinjiyuan Breeding Pig Co. Ltd., Tangshan) is collected, and the total RNA is extracted according to the operating procedure of the kit (commercially available from Bioteke Corporation, Catalog No. RP4002). The total RNA is reversely transcribed to cDNA according to the operating procedure of the kit (commercially available from TOYOBO Corporation, Catalog No. TRT-101), and the gene fragment of porcine growth hormone (GH) (the nucleotide sequence is presented as SEQ ID NO. 2 in the Sequence Listing) is amplified with the primers for amplifying GH as listed in Table 1. The reaction system is 50 μL, which contains 5 μL of 10xBuffer, 8 μL of 2.5 mM dNTP, 1 μL of 20 μM primer GH cDNA-L1, 1 μL of 20 μM primer GH-R1 (see Table 1 for the sequences), 0.5 μL of 5 U/μL high-fidelity Tag polymerase, and 100 ng of porcine cDNA as template, added up to 50 μL with ultra-pure water (the high-fidelity enzyme is commercially available from Takara (Dalian), Catalog No. DR010A). The procedure of PCR amplification: 98° C. for 10 s and 68° C. for 2 min, 30 cycles.

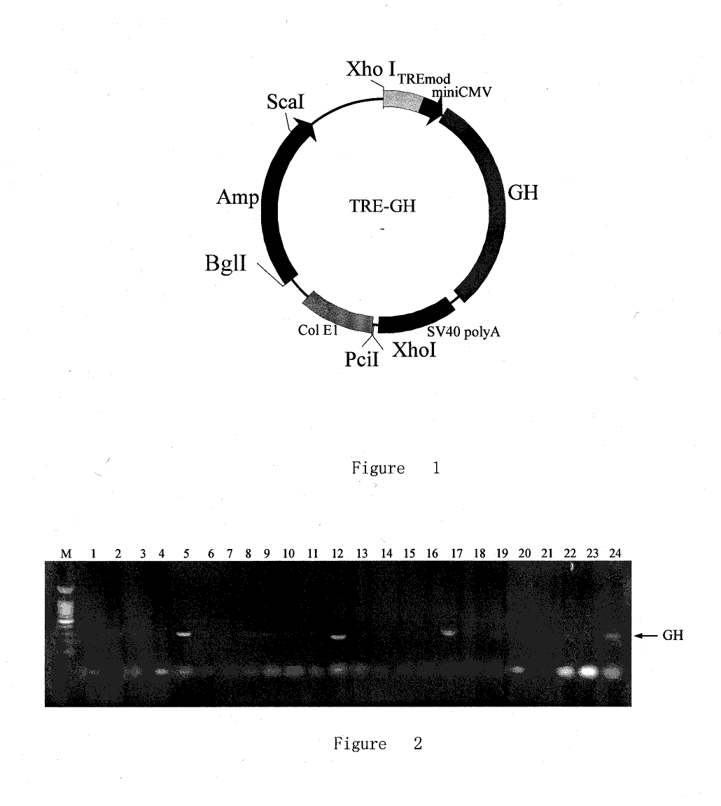

After the PCR amplified products are sequenced to be correct by sending to Invitrogen Beijing Office, the PCR products are recovered and purified using QIAGEN agarose gel kit (the operating steps are shown in the kit). The purified products are cleaved with two enzymes of KpnI and Mlul. Meanwhile, TRE vector prepared above is cleaved with two enzymes of KpnI and Mlu I. The PCR amplified products are linked to the cleaved vector and transformed (the operating steps and reagents are the same as above). Single colony is picked and sent to the corporation for sequencing; and the vector with correct sequencing result (named as TRE-GH) is selected for use (the profile for the vector is seen in FIG. 1).

The TET-ON vector of the invention is commercially available from clontech Corporation, Catalog No. 630930.

An endo-free plasmid maxiprep protocol (commercially available from OMEGA Corporation, Catalog No. D6948) is conducted on both of the plasmids TET-ON and TRE-GH vector described above. After the plasmid extraction and linearization by ScaI cleavage (commercially available from Fermanta Corporation, Catalog No. ER0431), the gene is purified and recovered according to the instruction (commercially available from QIAGEN Corporation, Catalog No. 12562). Hereto, linearized TRE-GH and TET-ON are obtained.

2. Obtainment of TRE-GH-TET-ON (as seen in FIG. 5)

The construction of linearized TRE-GH-TET-ON vector: TET-ON vector is cleaved with two enzymes of NheI (commercially available from NEB Corporation, Catalog No. R0131S) and HindIII (commercially available from NEB Corporation, Catalog No. R0104V), and the band at 2.5 Kb is recovered by gel. The TRE-GH vector of Step 1 is cleaved with two enzymes of NheI and HindIII at the same time, and the band at 3.3 Kb is recovered, which is linked to the fragments recovered above and transformed (the operating steps and reagents are the same as above). Single colony is picked, and a plasmid mini kit (commercially available from Bioteke Corporation, Catalog No. DP1002) is used to extract plasmid. The recombinant plasmid is identified by the cleavage with two enzymes of NheI and HindIII. The recombinant plasmid having identification result of cleavage and sequencing consistent with expectation is selected, and the endo-free plasmid maxiprep protocol is conducted. After the plasmid extraction and linearization by ScaI cleavage, the gene is purified and recovered according to the instruction to obtain the linearized TRE-GH-TET-ON vector.

### II. Obtainment of Transgenic Embryos

1. Injection of linearized TRE-GH and TET-ON into embryos

The liquid mixture containing linearized TRE-GH and TET-ON (molar ratio of 1:1) is injected into the pronuclei of the embryo of pigs (large white pig strain) in pronuclear stage by a conventional pronuclear-stage embryo microinjection method, and cultivated in NCSU 23 medium (commercially available from millipore Corporation, Catalog No. MR-182-D) until 8 cell stage.

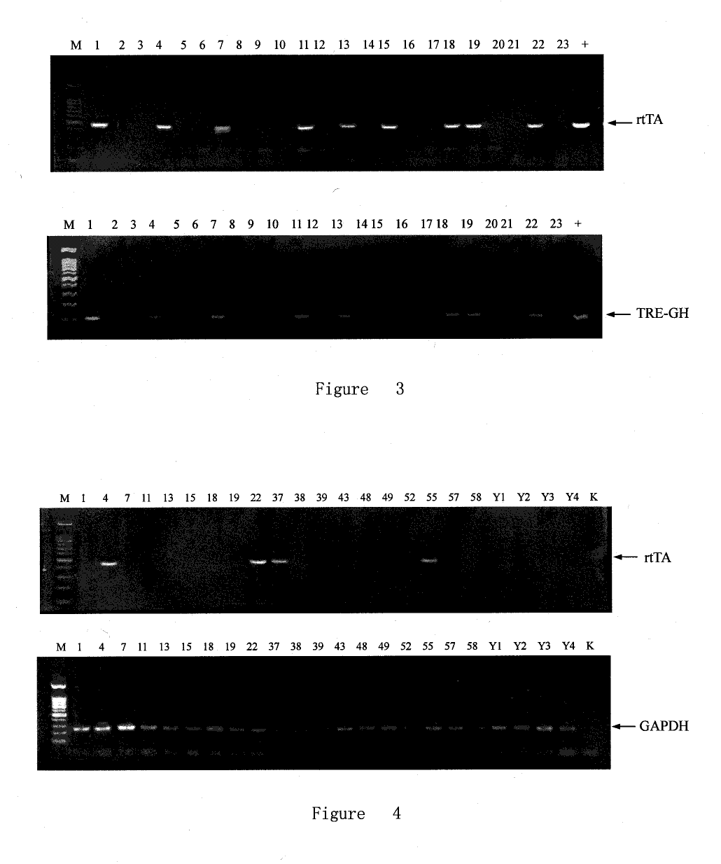

Normal embryos and the embryos microinjected with two genes are selected and 600 ng/ml of deoxytetracycline (DOX) is added into NCSU 23 liquid mediums. After 36 hours, an amplification reaction is performed using the embryo as template and according to the operating instruction of ProtoScript M-MuLV Taq RT-PCR Kit (commercially available from NEB Corporation, Catalog No. E6400S), 28 cycles (the primers are the quantitative primers for GH detection listed in Table 1). 5 μl of the PCR products are taken, on which 1% agarose gel electrophoresis is performed. Via the comparison of brightness of the electrophoretic bands, the expression level of GH is compared initially. As can be seen, compared to the GH expressed level of the normal embryos, the GH expressed levels in Embryo Nos. 3 and 5 of the embryos microinjected with two genes are significantly improved after the inducement of DOX (the result is shown in FIG. 2, M: mark; 1-10: are the GH expressions after the mixed genes of TRE-GH and TET-ON are microinjected followed by addition of deoxytetracycline; 11-20: are the GH expressions after that the mixed gene of TRE-GH-TET-ON is microinjected followed by addition of DOX; 21-24: are the GH expressions of normal embryos after addition of DOX).

2. Injection of Linearized TRE-GH-TET-ON into Embryos

The linearized TRE-GH-TET-ON is injected into the pronuclei of the embryo of pigs (large white pig strain) in pronuclear stage by microinjection method, and cultivated in NCSU 23 medium (commercially available from millipore Corporation, Catalog No. MR-182-D) until 8 cell stage.

Part of the normal embryos and embryos microinjected are selected and 600 ng/ml of DOX is added into NCSU 23 liquid medium. After 36 hours, the GH expression in embryos is detected directly using an embryo as template according to the operating instruction of ProtoScript M-MuLV Taq RT-PCR Kit (commercially available from NEB Corporation, Catalog No. E6400S) (the primers are the quantitative primers for GH detection listed in Table 1). As can be seen, compared to the GH expression level of the normal embryos, the GH level in Nos. 12 and 17 of the embryos microinjected with TRE-GH-TET-ON gene are significantly improved after the inducement of DOX (the result is shown in FIG. 2, M: mark; 1-10: are the GH expressions after the mixed genes of TRE-GH and TET-ON are microinjected followed by addition of deoxytetracycline; 11-20: are the GH expressions after that the mixed gene of TRE-GH-TET-ON is microinjected followed by addition of DOX; 21-24: are the GH expressions of normal embryos after addition of DOX).

### Example 2

### Obtainment of Transgenic Animals and the Detection Thereof

### I. Obtainment of Transgenic Animals

### 1. TRE-GH and TET-ON Transgenic Pigs

Step 1 of Step II in Example 1 is repeated, and linearized TRE-GH and TET-ON are injected into a porcine embryo in pronuclear stage and cultivated until 8 cell stage. Then, the embryo is transplanted into the uterine horn of an estrus synchronized sow through vaginal cervix in vitro.

According to the operating instruction of the kit (commercially available from Bioteke Corporation, Catalog No. DP1901), the genomic DNAs of the auricular tissues of the transgenic pigs are extracted, and then the pigs which are integrated with both TRE-GH and TET-ON simultaneously are screened by PCR. The reaction system of PCR is 50 μL, which contains 5 μL of 10xBuffer, 8 μL of 2.5 mM dNTP, 1 μL of 20 μM primer PL, 1 μL of 20 μM primer PR, and 0.5 μL of 5 U/μL high-fidelity Tag polymerase, added up to 50 μL with ultra-pure water. Using the porcine genomic DNA obtained as the template to be detected. The PCR amplification procedure is: 95° C. for 5 min; 94° C. for 20 s, and annealing temperature of 56° C., 72° C. for 1 min, 30 cycles, and final extension at 72° C. for min. PCR amplified products are detected using 1% agarose gel electrophoresis, and the positive pigs in which TRE-GH gene and rt-TA gene could be amplified respectively are screened out (1, 4, 7, 11, 13, 15, 18, 19 and 22 in FIG. 3). The detecting primers for the two genes are shown in Table 1 (the detecting primers are rtTA: rtTA-RTL1 and rtTA-RTR1; TRE-GH: TRE-L1 and TRE-L2 in Table 1, respectively).

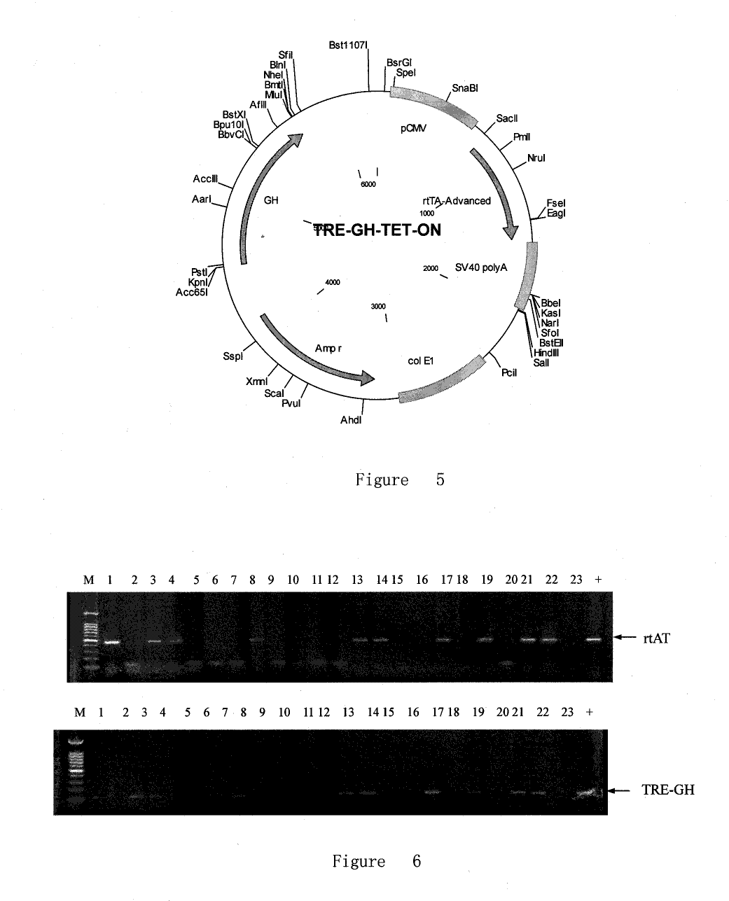

200 μL of blood is collected from the porcine auricular veins of the transgenic positive pigs aforementioned (1, 4, 7, 11, 13, 15, 18, 19 and 22), and the total RNA is extracted and reversely transcribed to cDNA (the operating steps and reagents are the same as above). Using porcine GAPDH gene as internal reference (the primers are mGAPDH-L1 and mGAPDH-R1), the semi-quantitative PCR is performed to measure rtTA expression. The loading system and reaction system of PCR are the same as those of the transgenic detection of rtTA (rtTA primers are rtTA-RTL1 and rtTA-RTR1 as shown in Table 1). The deoxytetracycline induction experiments on transgenic pigs are performed on Nos. 4 and 22 of the transgenic pigs capable of expressing rtTa as screened out (FIG. 4).

### 2. TRE-GH-TET-ON Transgenic Pigs

Step 2 of Step 11 in Example 1 is repeated, and linearized TRE-GH and TET-ON are injected into porcine embryos in pronuclear stage and cultivated until 8 cell stage. Then, the embryos are transplanted into the uterine horn of an estrus synchronized sow through vaginal cervix in vitro.

According to the operating instruction of the kit, the genomic DNAs of the auricular tissues of transgenic pigs are extracted (the reagents and operating steps are the same as above), and the pigs which are integrated with TRE-GH-TET-ON simultaneously are screened by PCR. The reaction system of PCR is 50 μL, which contains 5 μL of 10xBuffer, 8 μL of 2.5 mM dNTP, 1 μL of 20 μM primer PL, 1 μL of 20 μM primer PR and 0.5 μL of 5 U/μL high-fidelity Tag polymerase, added up to 50 μL with ultra-pure water. Using the porcine genomic DNA obtained as the template to be detected. The PCR amplification procedure is: 95° C. for 5 min; 94° C. for 20 s, annealing temperatures of 56° C. and 72° C. for 1 min, 30 cycles, and final extension at 72° C. for 5 min (detecting primers are rtTA: rtTA-RTL1 and rtTA-RTR1; TRE-GH: TRE-L1 and TRE-R1 in Table, respectively). PCR amplified products are detected using 1% agarose gel electrophoresis, and the positive pigs in which TRE-GH gene and rt-TA gene could be amplified respectively are screened out (37, 38, 39, 43, 48, 49, 52, 55, 57 and 58 in FIG. 6). The detecting primers of the two genes are shown in Table 1.

200 μL of blood is collected from the porcine auricular veins of the transgenic positive pigs (37, 38, 39, 43, 48, 49, 52, 55, 57 and 58), and the total RNA is extracted and reversely transcribed to cDNA (the operating steps and reagents are the same as above). Using porcine GAPDH gene as internal reference (the primers are mGAPDH-L1 and mGAPDH-R1), the semi-quantitative PCR is performed to measure rtTA expression (FIG. 4). The loading system and reaction system of PCR are the same as those of the transgenic detection of rtTA (rtTA primers are rtTA-RTL1 and rtTA-RTR1 as shown in Table 1). The deoxytetracycline induction experiments on transgenic pigs are performed on Nos. 37 and 55 of the transgenic pigs capable of expressing rtTA as screened out.

### II. The Deoxytetracycline Inducing Experiment on Transgenic Pigs

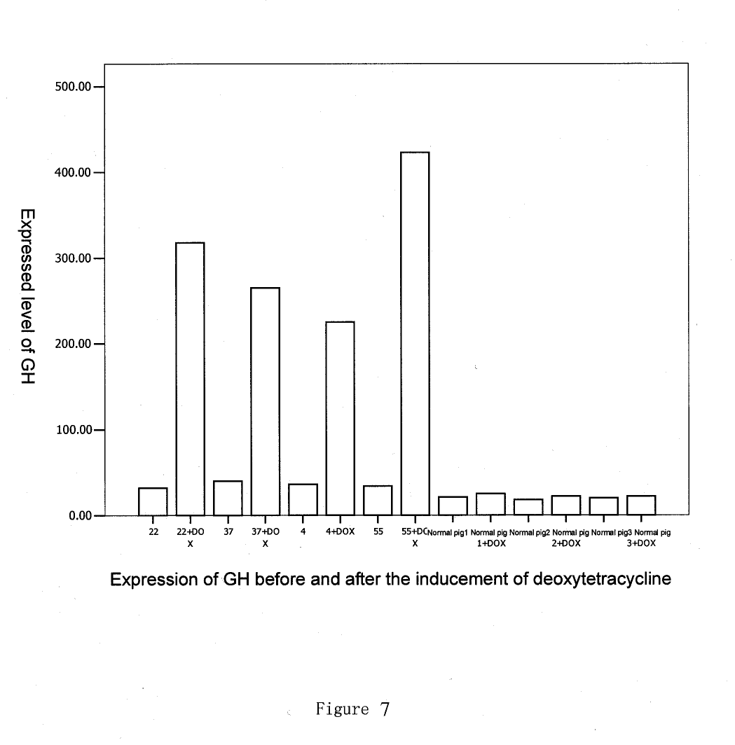

Deoxytetracycline inducing experiments are carried out on the transgenic pigs identified above (Nos. 4, 22, 37 and 55). Deoxytetracycline (commercially available from clontech Corporation, Catalog No. 631311, final concentration of 600 ng/ml) is added in drinking water. 200 μL of blood is collected via auricular veins 36 hours before or after the addition of deoxytetracycline, respectively, and the total RNA is extracted and reversely transcribed to cDNA (the operating steps and reagents are the same as above). Using porcine GAPDH gene as internal reference (the primers are mGAPDH-L1 and mGAPDH-R1 in Table 1), the real-time fluorescent quantitative PCR is performed to measure the expression amount of GH (GH primers are pGH-RTL1 and pGH-RTR1 as shown in Table 1). The loading system is loaded in accordance with the ABI quantitative reagent instruction, and the reaction system is 20 μL, which contains 10 μL of 2×Mixturer (TAKARA, Catalog No. DRR041A), 1 μL of 20 μM upstream primer, 1 μL of 20 μM downstream primer, 1 μL of cDNA, added up to 20 μL with ultra-pure water. PCR amplification procedure is: 95° C. for 5 min; 94° C. for 20 s, annealing temperature of 56° C. and 72° C. for 1 min, 40 cycles, and final extension at 72° C. for 5 min. The values of growth hormone expressions of three normal pigs are 21, 18 and 20 prior to deoxytetracycline addition respectively, and the GH expressions in blood are 25, 22 and 22 after deoxytetracycline (DOX) addition respectively, with no significant difference between the GH expression in blood prior to and after DOX addition (P<0.05). The values of GH expressions in bloods of Nos. 4, 22, 37 and 55 of transgenic pigs are 36, 32, 40 and 34 respectively before DOX addition, while they are 225, 318, 265 and 423 after DOX addition respectively, with GH sharply increased in blood of the transgenic pigs after addition of DOX (see FIG. 7). The result indicates that both of the separate and integrated tetracycline inducement system can be effective to prepare transgenic domestic animals.

TABLE 1
Primers used for amplifying vector components in
the present invention

| | | | Length | |
|---|---|---|---|---|
| Name of | | | of | Cleavage sites |
| amplified | Primer | | product | introduced at |
| fragment | symbol | primer sequence | (bp) | 5′-terminal |
| GH cDNA | GH | ATAAGCTTCCACCATGGCTG | 657 | Kpn I |
| | cDNA-L1 | CAGGCCCTCGGACC* | | (underlined) |
| | GH-R1 | CGTCTAGAACTAGAAGGCAC | | Mlu I |
| | | AGCTGCT | | (underlined) |
| rtTA | rtTA-RTR1 | CGTGGATAGCGGTTTGACTC | 482 | — |
| detection | rtTA-RTL1 | GGGCAGAAGTGGGTATGATG | | — |
| TRE-GH | TRE-L1 | AACCGTCAGATCGCCTGGAG | 100 | |
| detection | TRE-R1 | GCCATGGTGGAAGCTTATCG | | |
| GH | pGH-RTL1 | AACTGGCTGCTGACACCTAC | 220 | — |
| quantitative | pGH-RTR1 | TGAAGACCCTGCTGAGGAAC | | — |
| primers | | | | |
| quantitative | mGAPDH-L1 | TACACAGCCACTCAGAAGAC | 249 | — |
| PCR detection | mGAPDH-R1 | TTTCACAGCCTCCGTGATAG | | — |
| of Pig GAPDH | | | | |
| as internal | | | | |
| reference | | | | |

Note:
the italics part in the sequence with * in the table is kozak sequence.

### INDUSTRIAL APPLICATION

The experiments demonstrate that: the values of GH expression in blood of Nos. 4, 22, 37 and 55 of the transgenic pigs are 36, 32, 40 and 34 before DOX addition respectively, while the values are 225, 318, 265 and 423 after DOX addition respectively, with GH sharply increased in blood of transgenic pigs after adding DOX. The result indicates that both of the separate and integrated tetracycline inducement system can be effective to prepare transgenic domestic animals.

1. A method for cultivating a transgenic embryo with an increased expression amount of porcine growth hormone comprising:
introducing a recombinant expression vector of 1) or 2) below into a target embryo to obtain a transgenic embryo with a higher expression amount of porcine growth hormone than a target embryo in which the recombinant expression vectors of 1) and 2) are not introduced:
1) TRE-GH and TET-ON;
2) TRE-GH-TET-ON,
wherein, TRE-GH is a recombinant expression vector formed by inserting the growth hormone (GH) DNA fragment presented as SEQ ID NO. 2 in the Sequence Listing into a polyclonal site of vector TRE, and the vector TRE is a recombinant vector formed by inserting the DNA fragment presented as SEQ ID NO. 1 in the Sequence Listing into a polyclonal site of plasmid pUC19;
TRE-GH-TET-ON is a recombinant expression vector obtained by linking a fragment of 2.5 Kb in size obtained by the cleavage of TET-ON with two enzymes of NheI and HindIII to a fragment of 3.3 KB in size obtained by the cleavage of TRE-GH with two enzymes of NheI and HindIII.

2. A method according to claim 1, wherein the embryo is an embryo in pronuclear stage.

3. A method according to claim 1, wherein the embryo is an embryo of pig, cattle, sheep, cat, dog, rabbit or murine.

4. A method for cultivating a transgenic animal with an increased expression amount of porcine growth hormone comprising:
transplanting the transgenic embryo prepared by the method of claim 1 into the body of a female target animal to obtain a transgenic animal with a higher expression amount of porcine growth hormone than a target animal without transplantation.

5. A method according to claim 4, wherein the target animal is pig, cattle, sheep, cat, dog, rabbit or murine.
